(19) United States
(12) Patent Application Publication
(10) Pub. No.: US 2017/0296568 A1
(43) Pub. Date: Oct. 19, 2017

(54) SIRTUIN GENE POTENTIATOR, AND PHARMACEUTICAL PRODUCT, COSMETIC PRODUCT, AND FOOD PRODUCT USING SAME

(71) Applicants: MORISHITA JINTAN CO., LTD., Osaka (JP); KYUSHU UNIVERSITY, NATIONAL UNIVERSITY CORPORATION, Fukuoka (JP)

(72) Inventors: Yoshinori KATAKURA, Fukuoka (JP); Takeru SUYAMA, Osaka (JP); Norihisa NISHIDA, Osaka (JP)

(21) Appl. No.: 15/628,684

(22) Filed: Jun. 21, 2017

Related U.S. Application Data
(63) Continuation of application No. 14/427,312, filed on Mar. 11, 2015.

(30) Foreign Application Priority Data
Sep. 13, 2012 (JP) 2012-201899

(51) Int. Cl. A61K 31/7048; A61K 8/97; A61K 8/60; A61K 36/185; A23L 33/105; A61Q 19/00; A23L 2/52

(52) U.S. Cl. CPC A61K 31/7048; A61Q 19/007; A61K 8/97; A23L 2/52; A61K 36/185; A23L 33/105; A61K 8/60; A23V 2002/00; A61K 2800/78

(57) ABSTRACT

Disclosed are a method for potentiating a sirtuin gene in a living body, provided to the living body as a pharmaceutical product, a cosmetic product, or a food product including a sirtuin gene potentiator. The sirtuin gene potentiator of the present invention contains a given polyphenol and/or terpenoid as an active component. The polyphenol and/or the terpenoid can be contained in the form of a plant such as pomegranate or a plant extract thereof. The sirtuin gene potentiator of the present invention is available from a familiar material to avoid concerns such as side effects on the human body in advance. Such a sirtuin gene potentiator is useful as a novel material in the fields of pharmaceutical products, cosmetic products, and food products.

### CROSS-REFERENCE TO RELATED APPLICATION

The present application is continuation claiming benefit and priority under 35 U.S.C. §120 of U.S. application Ser. No. 14/427,312, filed 11 Mar. 2015, now , which is a U.S. National Stage Application under 35 USC §371 of International Application No. PCT/JP2013/074939, filed 13 Sep. 2013, published as WO 2014/042261 A1 on 20 Mar. 2014, which in turn claims priority to Japanese Application No. 2012-201899, filed 13 Sep. 2012, the entirety of each of which are incorporated herein by reference.

### TECHNICAL FIELD

The present invention relates to a sirtuin gene potentiator, and a pharmaceutical product, a cosmetic product, and a food product using the same, and more specifically relates to a sirtuin gene potentiator as easily available and with little concerns to cause side effects on the human body, and a pharmaceutical product, a cosmetic product, and a food product using the same.

### BACKGROUND ART

In studies of aging and lifetime control, the involvement of Sirtuin, Sir2, which has the activity of an NAD-dependent deacetylase, has been attracting attention. SIRTs 1 to 7 are known as mammalian homologs of yeast Sir2. In particular, SIRT1 has been considered to be involved in the control of enhancement of fat mobilization, suppression of nerve axonal degeneration, insulin secretion from β cells, gluconeogenesis in the liver, and the like, and to realize life prolongation through the control.

It has been attracting attention that a substance available from a familiar material is used for enhancing the activity of the sirtuin gene so as to provide a life prolongation effect for mammals. Examples of the material include a lactic acid bacterium and a component derived from a lactic acid bacterium (Patent Document 1).

However, a few types of substances capable of enhancing the activity of such sirtuin gene and available from a familiar material have been found, and, furthermore, it is hard to say that there is a sufficient number of types of substances having an excellent activity.

### PRIOR ART DOCUMENTS

### Patent Documents

Patent Document 1: Japanese Laid-Open Patent Publication No. 2008-195673

### SUMMARY OF INVENTION

### Problems to be Solved by the Invention

The present invention solves the above-described problems, and it is an object thereof to provide a sirtuin gene potentiator, which is available from a familiar material and has an excellent enhancing activity, and a pharmaceutical product, a cosmetic product, and a food product using the same.

### Means for Solving the Problems

The present invention provides a sirtuin gene potentiator including a polyphenol as an active component. The polyphenol is at least one compound selected from the group consisting of punicalin, punicalagin, urolithin A, eugeniin, tellimagrandin I, and an analog thereof.

The present invention also provides a sirtuin gene potentiator including a terpenoid as an active component. The terpenoid is at least one compound selected from the group consisting of cafestol, kahweol, glycyrrhizin, and an analog thereof.

In one embodiment, the polyphenol is contained in the form of a plant or a plant extract.

In a further embodiment, the plant or the plant extract is pomegranate or a pomegranate extract.

In another embodiment, the terpenoid is contained in the form of a plant or a plant extract.

The present invention further provides a pharmaceutical composition, comprising the sirtuin gene potentiator.

The present invention further provides a cosmetic composition, comprising the sirtuin gene potentiator.

The present invention further provides a food composition, comprising an isolated polyphenol and/or terpenoid, wherein the polyphenol is at least one compound selected from the group consisting of punicalin, punicalagin, urolithin A, eugeniin, tellimagrandin I, and an analog thereof, and the terpenoid is at least one compound selected from the group consisting of cafestol, kahweol, glycyrrhizin, and an analog thereof.

### Effects of Invention

According to the present invention, a functional material capable of enhancing the activity of the sirtuin gene as considered to be involved in life prolongation and anti-aging can be easily provided in large amounts. The sirtuin gene potentiator of the present invention is available from a familiar material to previously avoid concerns such as side effects on the human body.

### BRIEF DESCRIPTION OF DRAWINGS

FIG. 1 is a graph showing the effect on the hSIRT1 promoter activity when various polyphenols or terpenoids are added to Caco-2-hSIRT1p-EGFP cells.

FIG. 2 is a graph showing the expression level of the sirtuin gene hSIRT1 in Caco-2 cells after various polyphenols or terpenoids are added.

FIG. 3 is a graph showing the effect on the hSIRT1 promoter activity when various plants or plant extracts are added to Caco-2-hSIRT1p-EGFP cells.

FIG. 4 is a graph showing the EGFP fluorescence intensity in HaCaT-hSIRT1p-EGFP cells after various polyphenols or terpenoids are added.

### MODE FOR CARRYING OUT THE INVENTION

Hereinafter, the present invention will be described in detail.

A sirtuin gene potentiator according to the present invention contains a plant and/or a plant-derived component as an active component.

The term “sirtuin gene” used in the present invention may refer to, for example, homologs of Sir2 having an NAD-dependent deacetylase active enzyme as considered to contribute to life prolongation of yeast, Nematode worms, and Drosophila flies, and SIRT1, SIRT2, SIRT3, SIRT4, SIRT5, SIRT6 and SIRT7 in Mammals. The term “sirtuin gene potentiator” may refer to a substance itself capable of enhancing the activity of sirtuin gene in vivo and/or in vitro, and a composition containing that substance.

Examples of the plant in the present invention include grasses and trees such as pomegranate (Punica granatum); cinnamon (Cinnamomum sieboldi); barley (Hordeum vulgare); kale (Brassica oleracea acephala); ginseng (Panax ginseng); elder flower (Sambucus nigra); tea; rosemary (Rosmarinus officinalis); roses; cherry blossoms; ginger (Zingiber officinale); red ginger (Zingiber officinale var. rubrum); black ginger (Kaempferia parviflora); Strawberry Geranium (Saxifraga stolonifera); eucalypt (Eucalyptus globulus); evening primrose (Oenothera tetraptera); coffee (Coffea); liquorice (Glycyrrhiza glabra); and Chilean evening primrose (Oenothera stricta), and a combination thereof. Examples of the section of the plant that may be used include, but are not limited to, whole plants, flowers, fruits, leaves, seeds, roots, stems, rhizomes, root bark, and bark, and the sections fermented as necessary. Preferable examples of a combination of the specific exemplary plant and the preferably used section include flowers, fruits, leaves, and seeds of pomegranate, root bark and bark of cinnamon, young leaves of barley, fermented ginseng, fermented tea leaves (e.g., Oolong tea), leaves of rosemary, flowers, fruits, and seeds of roses, bark, flowers, and leaves of cherry blossoms, rhizomes of ginger, rhizomes of red ginger, rhizomes of black ginger, whole plants of Strawberry Geranium, leaves and bark of eucalypt, leaves of evening primrose, fruits of coffee, roots of liquorice, and whole plants of Chilean evening primrose.

The plant may be previously dried or may be raw (undried). The plant in use is preferably previously dried, in terms of having an excellent shelf life as a sirtuin gene potentiator and having an enhanced level of the active component in the potentiator.

The plant-derived component in the present invention may be an extract that is available from the plant (which may be either an extracted compound itself or an extracted mixture in the form of liquid, paste, powder, or the like), or any compound (e.g., chemically synthesized material) having the chemical structure similar to that of the extracted compound.

Examples of the plant-derived component include polyphenol and terpenoid, and a combination thereof. Examples of the polyphenol include punicalin, punicalagin, urolithin A, oenothein B, eucalbanin B, eugeniin, and tellimagrandin I, an analog thereof, and a combination thereof. Examples of the terpenoid include cafestol, kahweol, and glycyrrhizin, an analog thereof, and a combination thereof.

In the present invention, the plant-derived component can be obtained, for example, by extraction from a given section of the plant using a method known to those skilled in the art.

The extraction can be performed, for example, by immersing the section of the plant in a given extracting solvent.

In the immersion, for example, the plant may be previously cut into pieces with an appropriate length or pulverized in order to improve the extraction efficiency.

Examples of the extracting solvent include, but are not limited to, water (e.g., hot water); lower alcohols such as methanol, ethanol, propanol, and butanol; polyhydric alcohols such as propylene glycol and butylene glycol; ketones such as acetone and methyl ethyl ketone; esters such as methyl acetate and ethyl acetate; linear and cyclic ethers such as tetrahydrofuran and diethyl ether; hydrogen halides such as dichloromethane, chloroform, and carbon tetrachloride; hydrocarbons such as hexane, cyclohexane, and petroleum ether; aromatic hydrocarbons such as benzene and toluene; polyethers such as polyethylene glycol; and pyridines. These materials can be used alone or in a combination. The extracting solvent is preferably water, lower alcohol (methanol, ethanol, butanol, etc.), acetone, ethyl acetate, or a mixture liquid of two or more of these.

There is no particular limitation on the extraction conditions (the amount of solvent, the temperature, the time, etc.). For example, the extracting solvent is preferably in an amount of volume/dry mass that is 1 to 50 times that of the plant that is to be immersed therein. The extraction temperature may vary depending on the type of solvent used, but is typically set to a temperature that is at least room temperature and not greater than the boiling point of the solvent. The extraction time also may vary depending on the type of solvent used, the amount thereof, and the extraction temperature. For example, in use at room temperature, the extraction time may be 1 to 60 hours, and, in use at a temperature near the boiling point of the solvent, the extraction time may be approximately 1 to 300 minutes. Furthermore, the extraction may be performed once using one type of extracting solvent, or may be performed a plurality of times using different types of solvent.

After the immersion, the liquid is allowed to cool, for example, to room temperature, and the plant is removed therefrom by filtering or centrifugal separation. In this manner, a crude extract can be obtained. The obtained crude extract is purified using a suitable means (e.g., column chromatography) for removing impurities, and the solvent is removed therefrom.

With this processing, a desired plant-derived component can be obtained from the plant.

In one embodiment, the sirtuin gene potentiator of the present invention contains polyphenol (e.g., ellagitannin) as an active component. Examples of the ellagitannin include punicalin, punicalagin, urolithin A, oenothein B, eucalbanin B, eugeniin, tellimagrandin I, and an analog thereof (e.g., C1 to C20 saturated fatty acid ester or unsaturated fatty acid ester), and a combination thereof. The polyphenol may be contained in the form of a plant or a plant extract. If a plant itself is used, the whole plant or any given section described above may be used in a state where it is either raw or dried, or in the form of paste or powder obtained therefrom. If a plant extract is used, for example, an extract prepared as described above may be used. An example of the plant or the plant extract is pomegranate or a pomegranate extract. The pomegranate and the pomegranate extract may contain polyphenol such as ellagitannin (e.g., punicalin, punicalagin, urolithin A, oenothein B, eucalbanin B, and an analog thereof). The polyphenol such as ellagitannin (e.g., punicalin, punicalagin, urolithin A, oenothein B, eucalbanin B, and an analog thereof) may be contained in the form of pomegranate or a pomegranate extract. The eugeniin and an analog thereof may be contained in the form of roses or an extract thereof. The tellimagrandin I and an analog thereof may be contained in the form of Ramanas rose (Rosa rugose) or an extract thereof.

In one embodiment, the sirtuin gene potentiator of the present invention contains terpenoid as an active component. Examples of the terpenoid include cafestol, kahweol, glycyrrhizin, and an analog thereof (e.g., C1 to C20 saturated fatty acid ester or unsaturated fatty acid ester), and a combination thereof. The polyphenol may be a plant or a plant extract, or a combination thereof. The terpenoid may be contained in the form of a plant or a plant extract. The cafestol, the kahweol, and an analog thereof may be contained in the form of coffee or a coffee extract. The glycyrrhizin and an analog thereof may be contained in the form of liquorice or a liquorice extract.

The sirtuin gene potentiator of the present invention may be widely used generally for the purpose of enhancing the activity of the sirtuin gene, which may be used in vivo or in vitro. For example, the sirtuin gene potentiator of the present invention may be used as a constituent of a pharmaceutical composition such as a pharmaceutical product or a quasi-pharmaceutical product, alone or in a combination with another pharmaceutical composition, may be used as a constituent of a cosmetic composition such as a cosmetic product, alone or in a combination with any other cosmetic ingredient, may be used as a food composition such as a health food product or a type of additive that is to be added to foods and drinks, or may be used as a constituent of a feed composition used in the fields of livestock or cultured fish production, alone or in a combination with any other feed ingredient.

The sirtuin gene potentiator of the present invention may be made of the plant and/or the plant-derived component alone, that is, made only of the plant and/or the plant-derived component, or may further contain commonly used any other additive and component, as a component constituting the pharmaceutical composition, the cosmetic composition, the food composition, or the feed composition, as long as the plant and/or the plant-derived component is contained as an active component.

If the sirtuin gene potentiator of the present invention contains the plant and/or the plant-derived component and the above-mentioned other additive, there is no particular limitation on the proportion of the plant and/or the plant-derived component contained in the potentiator, but it is, for example, 0.01 to 99.99% by mass, preferably 1 to 90% by mass, with respect to the total mass of the potentiator.

Where the sirtuin gene potentiator of the present invention is used as a constituent of a pharmaceutical composition, the above-mentioned other additive may be an additive commonly used conventionally for preparing a pharmaceutical product. Examples of the additive include, but are not limited to, pharmaceutically acceptable vehicles, binders, disintegrant, lubricants, flavoring agents, colorants, and coating agents.

In another aspect, the present invention is directed to a pharmaceutical composition containing the sirtuin gene potentiator.

There is no particular limitation on the form of the pharmaceutical composition of the present invention, but typical examples thereof include various dosage forms for administration as defined in the Japanese Pharmacopoeia. In the case of a pharmaceutical composition for oral administration, examples of the form include tablet, capsule, powder, granule, fine granule, sustained-release tablet, solution, syrup, and emulsion. In the case of a pharmaceutical composition for parenteral administration, examples of the form include injection, ointment, and lotion. The dose of the pharmaceutical composition of the present invention varies depending on various conditions such as the body weight of a subject, and, thus, it may be selected as appropriate by those skilled in the art. In the pharmaceutical composition of the present invention, the amount of the active component of polyphenol or terpenoid, and combination thereof may be, for example, 0.1 to 50% by mass for administration through an oral route or by transmucosal absorption, and may be, for example, 0.1 to 30% by mass for parenteral administration.

Where the sirtuin gene potentiator of the present invention is used as a constituent of a cosmetic composition, the above-mentioned other additive may be any additive commonly used conventionally for preparing a cosmetic product. Examples of the additive include oils, surfactants, moisturizing agents, thickeners, antiseptics, flavoring substances, coloring matters, and medicaments. One, or two or more of these components may be contained as necessary.

In another aspect, the present invention is directed to a cosmetic composition containing the sirtuin gene potentiator.

Examples of the form of the cosmetic composition of the present invention include, but are not limited to, lotion, emulsion, cream, and powder.

Where the sirtuin gene potentiator of the present invention is used as a constituent of a food composition, the above-mentioned other component may be any food ingredient commonly used conventionally in the field of food products. Examples of the food ingredient include water; alcohols; edible meat products; ordinary food ingredients such as rice, wheat, corn, potato, sweet potato, soybean, kelp, wakame seaweed, and agar-agar, and powders thereof, sugars such as starch, cornstarch, starch syrup, lactose, fructose, dextrose, sucrose, sorbitol, and mannitol; dietary fibers such as apple fiber and soybean fiber; meat extracts; black vinegar extracts; gelatins; honeys; animal and vegetable fats and oils; spices; and food additives such as vitamins, preservative, dextrin, colorant, lubricant, emulsifier, suspending agent, antioxidant, antiseptic, thickener, sweetener, flavoring agent, polyvinylpyrrolidone, and crystalline cellulose. Other biologically active agents and medicaments (including Chinese herbal medicine) also may be contained as necessary. There is no particular limitation on the amount of such other agents and/or other medicaments, and those skilled in the art can select an appropriate component and amount that do not inhibit the activity of the sirtuin gene.

In another aspect, the present invention is directed to a food composition containing the sirtuin gene potentiator. The food composition of the present invention more preferably contains polyphenol and/or terpenoid isolated once from the plant, as the sirtuin gene potentiator.

Herein, “isolated” refers to the separating a component contained in the plant from that plant, through the procedure such as extraction as mentioned above. Thus, a composition in which the plant is directly contained as a constituent, for example, regardless of whether dried or undried, is not included as the food composition of the present invention.

As the food composition of the present invention contains so isolated polyphenol and/or terpenoid in this manner, it is possible to incorporate into the food composition a higher concentration of polyphenol and/or terpenoid (sirtuin gene potentiator), which is unattainable in the case where the plant is used directly as foods and drinks. As a result, it is possible to provide a food composition that is completely different from conventional foods and drinks.

The food composition of the present invention generally refers to those typically used as food, and there is no limitation on the form thereof. The food composition is not limited to a solid food product, and may be a beverage (e.g., liquid beverage).

Herein, the term “food composition” used herein generally refers to food products regardless of whether or not they require mastication for ingestion, and examples of the form thereof include paste, solid (including tablet and granule), jelly, and liquid, at ambient temperature. Specific examples of the food composition include, but are not limited to, confectionery such as candies, gummy candies, cookies, and biscuits; syrups; fruit and vegetable products such as dry fruits and dry vegetables; pickled vegetables such as pickled radish and Kimchi; meat and fish products such as beef jerky, hamburg steaks, hams, and sausages; noodles such as Chinese noodles, wheat noodles, buckwheat noodles, pasta, and thin wheat noodles; breads such as sliced breads, French breads, buns stuffed with sweet bean paste, and stuffed buns; rice cakes such as rice cakes stuffed with sweet filling and rice cakes flavored with mugwort; canned or bottled foods such as canned fruits; jellies; ice creams; supplements such as nutritional supplement foods; and beverages such as fruit beverages, tea beverages, coffee beverages, milk beverages, alcohol beverages, and soft drinks.

As described above, the sirtuin gene potentiator of the present invention can enhance the activity of the sirtuin gene. Accordingly, the sirtuin gene potentiator can be widely used as a material that realizes or is expected to provide the life prolongation or longevity effect for the living body.

### EXAMPLES

Hereinafter, the present invention will be more specifically described by way of examples, but the invention is not limited to the following examples.

### Reference Example 1

Cells into which the promoter of the sirtuin gene was introduced were prepared as shown below in order to use them in screening for the sirtuin gene potentiator.

First, the promoter region (−1593 to −1 bp) of hSIRT1 (human SIRT1) was acquired using the LA-PCR method using, as a template, a human genome DNA extracted from TIG-1 cells (obtained from Institute of Development, Aging and Cancer, Tohoku University), with primers synthesized based on reported information of the hSIRT1 genomic sequence so as to synthesize primers having added AseI and NheI recognition sequences at their respective ends (hSIRT1p-AseI (SEQ ID NO: 1) and hSIRT1p-NheI (SEQ ID NO: 2)). The PCR reaction condition was at 94° C. for 1 minute, followed by at 98° C. for 20 seconds; and at 68° C. for 2 minutes, 34 cycles; and then at 72° C. for 10 minutes for extension reaction. LA-Taq from Takara Shuzo Co., Ltd. was used as the TaqDNApolymerase. The primers were synthesized by Nippon EGT based on our request.

The hSIRT1 promoter fragment obtained by the LA-PCR was subjected to TA cloning using a pGEM-T Easy vector (manufactured by Promega KK). Furthermore, the base sequence thereof was confirmed by sequencing. The hSIRT1 promoter fragment, inserted into the pGEM-T Easy vector, was cut out by digestion with the restriction enzymes AseI and NheI, and the CMV promoter of a pEGFP-C3 (manufactured by Takara Bio Inc.) was removed by digestion with AseI and NheI in a similar manner, and the hSIRT1 promoter fragment was inserted into that removed site, so that phSIRT1p-EGFP was obtained.

Transfection was performed using a LIPOFECT AMINE 2000 REAGENT (manufactured by Life Technologies) according to its protocol. On the day before the transfection, Caco-2 cells (human colonic cancer-derived cells obtained from Riken BioResource Center) were plated at 1.0×10⁶ in a 10-mL dish. On the day of the transfection, phSIRT1p-EGFP (10 μg) was diluted in a serum-free OPTI-MEM medium to a volume of 750 μL, after which 15 μL of LIPOFECT AMINE Reagent was diluted in a serum-free OPTI-MEM medium to 1.5 mL, and they were incubated at room temperature for 5 minutes. The transfection using the LIPOFECT AMINE Reagent was performed in the absence of serum, during which the culture medium of the Caco-2 cells plated on the previous day was removed, and was replaced by 5 mL of serum-free OPTI-MEM medium. After 30 minutes of this, 1.5 mL of DNA-LIPOFECT AMINE 2000 mixture was added to each 10-mL dish. After the addition, the dish was gently shaken to mix the DNA-LIPOFECT AMINE and the OPTI-MEM. After the culture for 3 hours, the DNA-LIPOFECT AMINE mixture solution was removed, and the medium was replaced by a serum-containing culture medium. Subsequently, culture was performed under the environment at 5% CO₂/95% air and 37° C. for 21 hours, and the culture medium was replaced by a new one.

Since phSIRT1p-EGFP is resistant to the drug G418, the drug G418 was added to the transfected cells to 70 μg/mL to subject them to the drug selection for a week. The culture medium was replaced by a new one every three days, and G418 was added every time at the same concentration. Accordingly, Caco-2-hSIRT1p-EGFP cells were obtained.

### Example 1

### Screening for SIRT1 Enhancing Polyphenol or Terpenoid

In this example, Caco-2-hSIRT1p-EGFP cells were used to examine the effect of various polyphenols or terpenoids on the enhancement of the sirtuin gene hSIRT1 promoter.

Caco-2-hSIRT1p-EGFP cells were plated at 0.6×10⁴ cells/well in a 96-well plate. On the next day, 10 μM polyphenol or terpenoid, or control (PBS) was added to each well. At 2 days after the addition, the culture solution was aspirated, after which 100 μL of 4% paraformaldehyde was added to each well, and the plate was allowed to stand at room temperature for 10 minutes. After the plate was allowed to stand for 10 minutes, 4% paraformaldehyde was aspirated, 100 μL of Cellstain (registered trademark)-Hoechst 33342 solution (Dojindo) diluted 1/500 with PBS was added to each well, and the plate was allowed to stand at room temperature in the dark for 15 minutes and the solution was then aspirated. Then, 100 μL of PBS was added to each well, and the fluorescence intensity was measured using an IN Cell Analyzer 1000 (GE Healthcare, Amersham Place, UK). The promoter activity was represented by a relative ratio to the activity of the control.

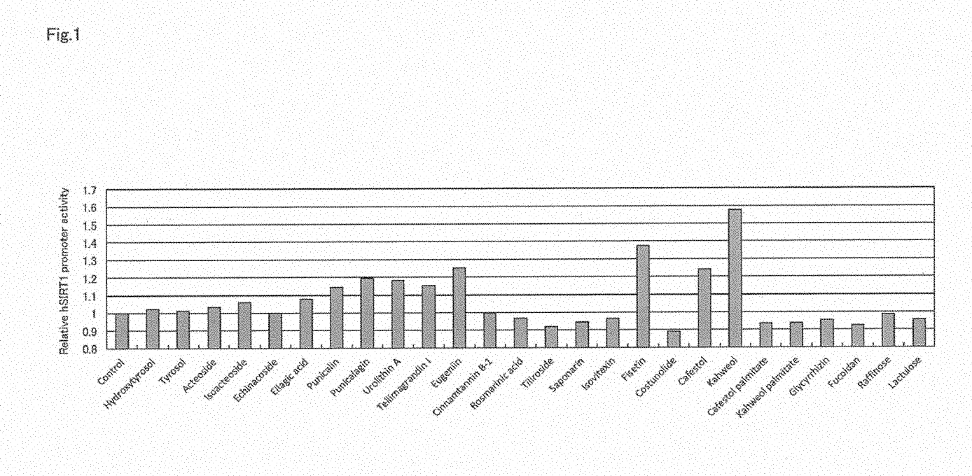

The results are shown in FIG. 1. FIG. 1 is a graph showing the effect on the hSIRT1 promoter activity when various polyphenols or terpenoids are added to Caco-2-hSIRT1p-EGFP cells. The vertical axis indicates a relative hSIRT1 promoter activity, where a higher value means a higher activity of promoter. On the horizontal axis, the polyphenol or terpenoid used are shown. A particularly strong hSIRT1 promoter enhancing activity was seen for punicalin, punicalagin, urolithin A, tellimagrandin I, eugeniin, cafestol, kahweol, and fisetin.

### Example 2

### Investigation of Expression Level of Endogenous SIRT1 Obtained by SIRT1 Enhancing Polyphenol or Terpenoid

In this example, the expression level of the sirtuin gene hSIRT1 in Caco-2 cells after polyphenol or terpenoid, namely punicalin, punicalagin, urolithin A, eugeniin, cafestol, kahweol, glycyrrhizin, or fisetin was added. The procedure is shown below.

Caco-2 cells were plated at 3.0×10⁵ cells for a 5-mL dish. After 24 hours, each polyphenol was added to be 10 μM. Note that as a negative control, none of polyphenol nor terpenoid was added, and as a positive control, 10 μM resveratrol was added. At 2 days after this treatment, RNA was collected.

The total RNA was prepared using High Pure RNA Isolation Kit (manufactured by Roche, Indianapolis, Ind., USA). For the procedures from the total RNA preparation to the reverse transcription, the reagents and tools used were RNase-free. Cells were prepared in a sub-confluent to confluent condition in a cell culture dish (Greiner bio-one, Monroe, N.C., USA). The medium was completely removed, 200 μL of PBS, and 400 μL of cell lysis solution, which was contained in High Pure RNA Isolation Kit, were added, and the entire dish was well covered by the cell lysis solution. When the viscosity of the cell lysis solution was decreased, the cell lysis solution was collected into a 1.5-mL sample tube. The collected sample was well suspended. A filter tube and a collection tube, which were contained in High Pure RNA Isolation Kit, were assembled, and the sample was pipetted into a buffer reservoir in the upper portion of the filter tube, and was centrifuged at 8,000×g for 15 seconds. The flowthrough liquid into the collection tube was discarded, and the filter tube and the collection tube were again assembled. Then, 90 μL of DNase incubation buffer per sample was pipetted into a sterile reaction tube, and 10 μL of DNase I was added into the tube and mixed. The mixture liquid was pipetted into the buffer reservoir in the upper portion of the filter tube, was added on the glass fiber fleece in the filter tube, and was incubated at room temperature for 15 minutes. Then, 500 μL of wash buffer I, which was contained in High Pure RNA Isolation Kit, was added to the buffer reservoir in the upper portion of the filter tube, and was centrifuged at 8,000×g for 15 seconds. The flowthrough liquid into the collection tube was discarded, and the filter tube and the collection tube were again assembled. Then, 500 μL of wash buffer II was added to the buffer reservoir in the upper portion of the filter tube, and centrifugation was performed at 8,000×g for 15 seconds. The liquid discharged to the collection tube was discarded, and the filter tube and the collection tube were again assembled. Then, 200 μL of wash buffer II was added to the buffer reservoir in the upper portion of the filter tube, and centrifugation was performed at 13,000×g for 2 minutes, after which a remaining wash buffer in the filter tube was removed. The collection tube was discarded, the filter tube was inserted into a sterile reaction tube, and 50 μL of elution buffer was added in the filter tube, and centrifugation was performed at 8,000×g for 1 minute. The eluate obtained by the procedures was taken as an RNA solution. The concentration of RNA in the solution was calculated based on the light absorbance at 260 nm with a NanoDrop 2000/2000c spectrophotometer (Thermo Scientific, Waltham, Mass., USA), for use in subsequent experiments.

Then, 5 pmol of oligo(dT)₂₀ primer was added to 1.0 μg of total RNA extracted from the cells, and sterile water was further added thereto so that the total liquid amount was 13 μL. Heat treatment was performed using a thermal cycler (Peltier Thermal Cycler PTC-200, MJ Research, Watertown, Mass., USA) at 65° C. for 5 minutes, and the resultant was immediately transferred in ice to quench it. During this treatment, the reverse transcriptase reaction program was advanced to the 42° C. stage and was temporarily stopped. A mixture solution obtained by mixing 4 μL of reverse transcriptase reaction buffer solution, 2 μL of 1 mM dNTPs (Amershan Pharmacia Biotech., Buckinghamshire, UK), and 0.5 μL of reverse transcriptase ReverTra Ace (TOYOBO, Osaka, Japan) per sample was added to and gently mixed with the sample on ice after 5 minutes. Then, cDNA was synthesized through the reaction at 42° C. for 20 minutes and at 99° C. for 5 minutes, and was used as a template in subsequent PCR.

A forward primer (SEQ ID NO: 3) and a reverse primer (SEQ ID NO: 4) for hSIRT detection and a forward primer (SEQ ID NO: 5) and a reverse primer (SEQ ID NO: 6) for β-actin detection as primers for calibration curve were designed. The primers were synthesized by Takara Bio Inc. (Shiga, Japan) based on our request.

The prepared cDNA was used as a template. The cDNA was diluted to 1/10, and both the forward primer and the reverse primer were diluted to 10 pmol/μL. These dilutions were mixed such that 51.5 μL of RNase-free water, 3.5 μL of forward primer and 3.5 μL of reverse primer, 7.0 μL of template cDNA, and 22 μL of KAPA SYBR FAST qPCR Kit (NIPPON Genetics, Tokyo, Japan) were placed in a 0.2-mL PCR tube and were well suspended. Subsequently, the resultant was added at 25 μL/well in a 96-well plate, and was subjected to quantitative PCR using a Thermal Cycle Dicer Real Time System (TaKaRa). The PCR reaction condition was at 95° C. for 30 seconds, 1 cycle; and then at 95° C. for 5 seconds and at 60° C. for 30 seconds, 40 cycles. The relative gene expression level of hSIRT was determined by dividing the measured value by the value for β-actin.

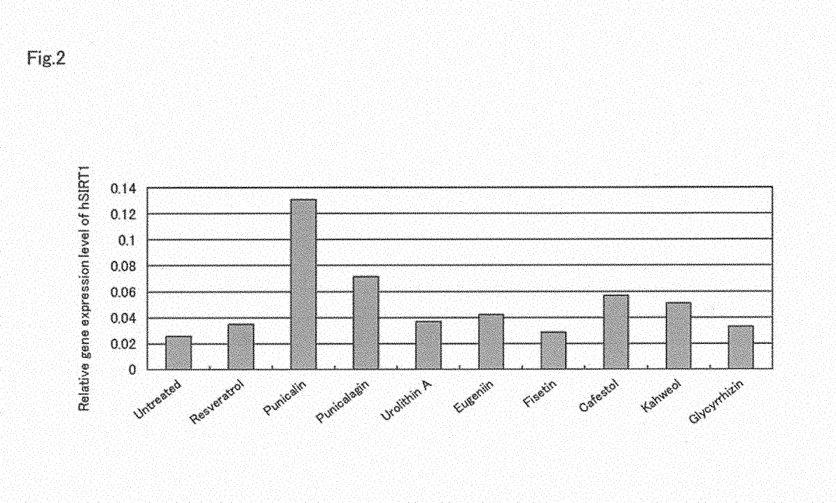

The results are shown in FIG. 2. FIG. 2 is a graph showing the expression level of the sirtuin gene hSIRT1 in Caco-2 cells after various polyphenols or terpenoids are added. The vertical axis indicates a relative gene expression level of hSIRT, and on the horizontal axis, the polyphenols or terpenoids used are shown. The hSIRT1 transcription enhancing effect was observed for all of punicalin, punicalagin, urolithin A, eugeniin, cafestol, kahweol, glycyrrhizin, and fisetin.

### Example 3

### Screening for SIRT1 Enhancing Plant or Plant Extract

In this example, the effect of enhancing the sirtuin gene hSIRT1 promoter was examined as in Example 1 except that various plants or plant extracts having various polyphenols or terpenoids were used.

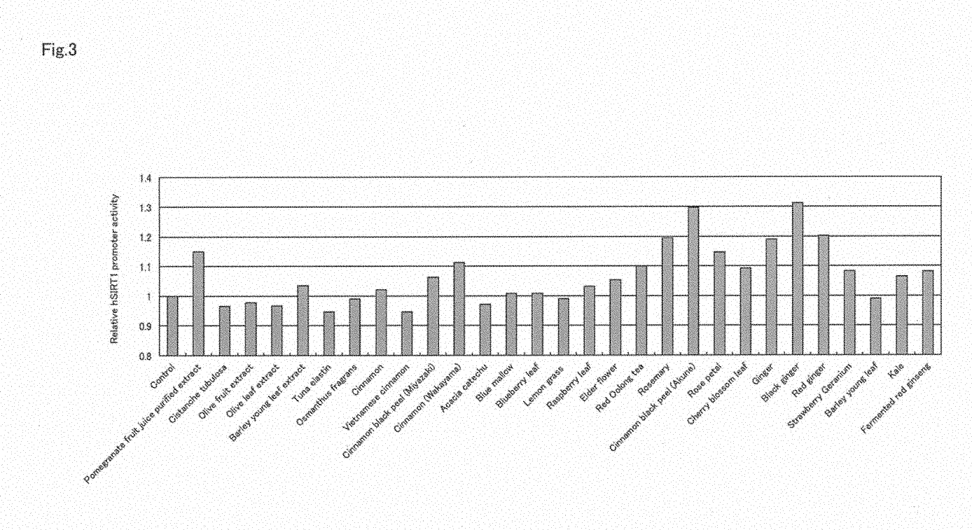

The results are shown in FIG. 3. FIG. 3 is a graph showing the effect on the hSIRT1 promoter activity when various plants or plant extracts are added to Caco-2-hSIRT1p-EGFP cells. The vertical axis indicates a relative hSIRT1 promoter activity, where a higher value means a higher activity of promoter. On the horizontal axis, the plant or plant extract used are shown. The hSIRT1 promoter enhancing activity was seen for pomegranate (Punica granatum) fruit juice purified extract, cinnamon (Cinnamomum sieboldi), cinnamon black peel (Miyazaki), cinnamon (Wakayama), elder flower (Sambucus nigra), red Oolong tea, rosemary (Rosmarinus officinalis), cinnamon black peel (Akune), rose petal, cherry blossom leaf, ginger (Zingiber officinale), black ginger (Kaempferia parviflora), red ginger (Zingiber Officinale var. rubrum), and Strawberry Geranium (Saxifraga stolonifera).

### Reference Example 2

In order to further investigate the SIRT1 expression enhancing effect of the sirtuin gene potentiator, cells into which a sirtuin gene promoter was introduced were prepared as shown below.

1. Cell Culture

HaCaT cells (human epidermis keratinocyte-derived cell lines, provided by Dr. Takumi Miura, National Center for Child Health and Development) were cultured using a Dalbecco's Modified Eagle Medium (DMEM) medium (Nissui Pharmaceutical Co., Ltd, Tokyo) supplemented with 10% FBS, 100,000 U/L penicillin (Meiji, Tokyo), 100 mg/L streptomycin (Meiji), and 2.0 g/L NaHCO₃. These cells were subcultured at 37° C. in the presence of 5% CO₂.

2. Gene Introduction by Lipofection

HaCaT cell lines (HaCaT-hSIRT1p-EGFP cells) into which the vector (phSIRT1-EGFP) described in Reference Example 1 was integrated were prepared as below.

2-1. Gene Introduction into HaCaT cells

The HaCaT cells were disseminated at 9.0×10⁵ in a φ60-mm dish, and were cultured in 10% FBS supplemented DMEM medium. Cells as a control were prepared in a similar manner. After 24 hours, 8 μg of phSIRT1p-EGFP was added to and mixed with 300 μL of DMEM medium, 24 μL of Hilymax (manufactured by Dojindo Laboratories), which is a transfection reagent, was further added and mixed therewith, and the mixture was incubated for 15 minutes at room temperature. Subsequently, the total amount was added to the HaCaT cells. After 3 hours, the medium was replaced by 10% FBS supplemented DMEM medium.

2-2. Drug Selection

At 24 hours after the gene introduction, the medium was replaced by 10% FBS supplemented DMEM medium containing 750 μg/mL of G418 (Wako Pure Chemical Industries, Ltd.). After visually confirming under a microscope that all the control non-treated HaCaT cells were killed, the subculture was continued using 10% FBS supplemented DMEM containing 150 μg/mL of G418.

2-3. Measurement of EGFP Fluorescence Intensity by Flow Cytometry

It was confirmed with flow cytometry that the phSIRT1p-EGFP vector was reliably transferred inro the cells after the subculture. The above-described cells and the control non-treated HaCaT cells were plated at 7.0×10⁵ per a φ60-mm dish, respectively. After 24 hours, the cells were picked up and well suspended in 2 mL of 5% FBS supplemented DMEM medium, and were filtered through a Nylon Mesh (K_{yos}hin Rikoh Inc., Japan), and the EGFP fluorescence intensity thereof was measured with a Flow Cytometer (EPICS, BeckmanCoulter). The analysis was performed using software FlowJo (Tree Star, Ashland Oreg.), so that the EGFP fluorescence intensity of each cell was represented as a histogram.

### Example 4

### Effect of Enhancing the Sirtuin Gene hSIRT1 Promoter by SIRT1 Enhancing Polyphenol or Terpenoid

HaCaT-hSIRT1p-EGFP cells were plated at 1.7×10⁶ cells in a φ60-mm dish (as measured by flow cytometry) or at 2.0×10⁴ cells/well in a 96-well plate (as measured by an IN Cell Analyzer 1000). After 24 hours, 10 μM of each polyphenol or terpenoid dissolved in DMSO (Wako Pure Chemical Industries, Ltd.) was added thereto. Note that as a negative control, the same amount of DMSO was added without polyphenol or terpenoid, and as a positive control, 10 μM resveratrol was added. At 24 hours after the addition of the various polyphenols or terpenoids or the controls, the fluorescence intensity was measured by flow cytometry.

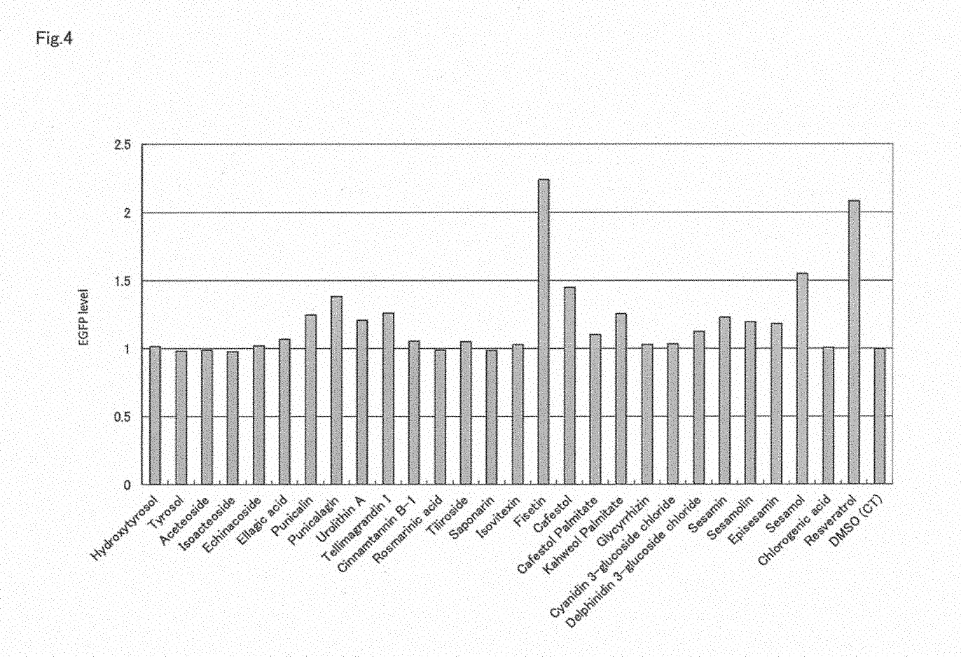

The results are shown in FIG. 4. FIG. 4 is a graph showing the EGFP fluorescence intensity in HaCaT-hSIRT1p-EGFP cells after various polyphenols or terpenoids are added. The vertical axis indicates an EGFP fluorescence intensity, where a higher value means a higher activity of promoter. On the horizontal axis, the polyphenol or terpenoid used are shown. The hSIRT1 expression effect on skin epidermis cells was seen for punicalin, punicalagin, urolithin A, tellimagrandin, fisetin, cafestol (derivative), and kahweol.

### Preparation Example 1

### Preparation of Punica granatum extract

After 700 mL of water was added to 300 g of commercially available Punica granatum dry powders (from fruits and seeds, made in China), the mixture was left with stirring at 50° C. for 24 hours, and was allowed to cool, followed by centrifugation to obtain 900 mL of extract. The extract was injected into a column filled with 100 g of Amberlite XAD4 (manufactured by Organo Corporation), 3000 mL of water was run therethrough, after which 1500 mL of mixture of ethanol:water=8:1 (v:v) was run therethrough. The obtained fraction was concentrated under reduced pressure, and the obtained ethanol-water fraction concentrate was freeze dried with 5 g of cellulose (AVICEL, manufactured by Asahi Kasei Corporation) as a freeze drying aid. In this manner, a Punica granatum powdered extract was prepared.

Ellagitannin Amount

The ellagitannin amount of the Punica granatum powdered extract was determined with HPLC (Model: Inertsil ODS-3, manufactured by GL Sciences Inc.) according to the conditions below as described in the document (J. Agric. Food Chem., 2009, 57(16), p. 7395).

HPLC Analysis Condition

Detector: Ultraviolet absorptiometer (380 nm)

Column: Inertsil ODS-3 (5 μm, 4.6×250 mm) (manufactured by GL Sciences Inc.)

Column temperature: 40° C.

Flow rate: 1.0 mL/min

Poured amount: 25 μL

Mobile phase condition: 0.5% phosphoric acid (A) and acetonitrile (B) were subjected to linear gradient according to the conditions below.

| | A | B |
|---|---|---|
| 0 min | 95% | 5% |
| 10 min | 85% | 15% |
| 30 min | 75% | 25% |
| 35 min | 95% | 5% |

The obtained results are shown below.

| | |
|---|---|
| Punicalin | 25% |
| Punicalagin | 30% |
| Oenothein B | 0.1% |

### Example 5

### Preparation of Tablets

In this example, 10 mg of pomegranate (Punica granatum) powdered extract of Preparation Example 1, 250 g of lactose, 45 g of cornstarch, and 20 g of carboxymethylcellulose calcium were placed in an oscillating granulator, were preheated and mixed, and 34 g of aqueous solution containing 1.7 g of hydroxypropylcellulose was sprayed thereon so as to obtain granulated powders. Then, 100 g of carboxymethylcellulose calcium and 40 g of talc were added and mixed therewith, and the mixture powders were compressed into tablets using a tablet machine, so as to obtain uncoated tablets

### Example 6

### Preparation of Beverage

A beverage was prepared according to the formulation below.

| Component | Mix (ratio by mass) |
|---|---|
| Glycerin | 10.0 |
| Pomegranate powdered extract of | 1.0 |
| Preparation Example 1 | |
| Cellulose | 0.1 |
| Citric acid | 0.3 |
| Flavoring substance | 0.1 |
| Purified water | Rest |

These components were mixed and stirred, so that a beverage was prepared.

### Example 7

### Preparation of Skin Lotion

The components below in the following proportion were uniformly mixed, so as to obtain a skin lotion.

| Component | Mix (ratio by mass) |
|---|---|
| Glycerin | 10.0 |
| 1,3-butylene glycol | 6.0 |
| Pomegranate powdered extract of | 1.0 |
| Preparation Example 1 | |
| Citric acid | 0.1 |
| Sodium citrate | 0.3 |
| Polyoxyethylene | 1.0 |
| Ethyl alcohol | 8.0 |
| Paraben | 0.1 |
| Flavoring substance | 0.1 |
| Purified water | Rest |

### Industrial Applicability

According to the present invention, a functional material capable of enhancing the activity of the sirtuin gene as considered to be involved in life prolongation and anti-aging can be easily provided in large amounts. The sirtuin gene potentiator of the present invention is available from a familiar material to previously avoid concerns such as side effects on the human body. Such a sirtuin gene potentiator is useful as a novel material in the fields of pharmaceutical products, cosmetic products, and food products.

1. A method for potentiating a sirtuin gene in a living body, comprising:
administering a polyphenol into the living body, wherein the polyphenol is at least one compound selected from the group consisting of punicalin, punicalagin, urolithin A, eugeniin, tellimagrandin I, and C1 to C20 saturated fatty acid ester or unsaturated fatty acid ester thereof.

2. A method for potentiating a sirtuin gene in a living body, comprising:
administering a terpenoid into the living body, wherein the terpenoid is at least one compound selected from the group consisting of cafestol, kahweol, glycyrrhizin, and C1 to C20 saturated fatty acid ester or unsaturated fatty acid ester thereof.

3. The method according to claim 1, wherein the polyphenol is contained in the form of a plant or a plant extract.

4. The method according to claim 3, wherein the plant or the plant extract is pomegranate or a pomegranate extract.

5. The method according to claim 2, wherein the terpenoid is contained in the form of a plant or a plant extract.

6. The method according to claim 1, wherein the polyphenol is contained in a pharmaceutical composition.

7. The method according to claim 1, wherein the polyphenol is contained in a cosmetic composition.

8. The method according to claim 1, wherein the polyphenol is contained in a food composition.

9. The method according to claim 2, wherein the terpenoid is contained in a pharmaceutical composition.

10. The method according to claim 2, wherein the terpenoid is contained in a cosmetic composition.

11. The method according to claim 2, wherein the terpenoid is contained in a food composition.


SEQUENCE LISTING (6 sequences)

SEQ ID NO: 1 - DNA, 30 bp, Artificial
  hSIRT1-f-AseI
aattattaatacttcaggtgatctgtccgc

SEQ ID NO: 2 - DNA, 30 bp, Artificial
  hSIRT1-f-NheI
aattgctagccttccaactgcctctctggc

SEQ ID NO: 3 - DNA, 24 bp, Artificial
  forward primer for hSIRT1 detection
gcctcacatgcaagctctagtgac

SEQ ID NO: 4 - DNA, 24 bp, Artificial
  reverse primer for hSIRT1 detection
ttcgaggatctgtgccaatcataa

SEQ ID NO: 5 - DNA, 19 bp, Artificial
  forward primer for beta-actin detection
tggcacccagcacaatgaa

SEQ ID NO: 6 - DNA, 25 bp, Artificial
  reverse primer for beta-actin detection
ctaagtcatagtccgcctagaagca